(19) United States
(12) Patent Application Publication
(10) Pub. No.: US 2013/0243872 A1
(43) Pub. Date: Sep. 19, 2013

(54) PHARMACEUTICAL COMPOSITE FORMULATION COMPRISING HMG-COA REDUCTASE INHIBITOR AND ASPIRIN

(71) Applicants: Yong Il Kim, Suwon-si (KR); Young Jun Na, Gwacheon-si (KR); Jun Young Choi, Suwon-si (KR); Min Jung Kim, Gunpo-si (KR); Jae Hyun Park, Suwon-si (KR); Jong Soo Woo, Suwon-si (KR)

(72) Inventors: Yong Il Kim, Suwon-si (KR); Young Jun Na, Gwacheon-si (KR); Jun Young Choi, Suwon-si (KR); Min Jung Kim, Gunpo-si (KR); Jae Hyun Park, Suwon-si (KR); Jong Soo Woo, Suwon-si (KR)

(73) Assignee: HANMI SCIENCE CO., LTD, Hwaseong-si, Gyeonggi-do (KR)

(21) Appl. No.: 13/988,947

(22) PCT Filed: Dec. 14, 2011
(86) PCT No.: PCT/KR11/09628

§ 371 Date: May 22, 2013

(30) Foreign Application Priority Data
Dec. 17, 2010 (KR) 10-2010-0129838

(51) Int. Cl. A61K 9/16; A61K 31/40; G01N 33/15; A61K 31/616

(52) U.S. Cl. CPC A61K 9/16; A61K 31/616; A61K 31/40; G01N 33/15
USPC 424/495; 424/494; 514/165; 73/866

(57) ABSTRACT

Provided is a pharmaceutical composite formulation for preventing or treating cardiovascular diseases, which comprises (1) a first particle comprising an HMG-CoA reductase inhibitor and a basic additive; and (2) a second particle comprising a core containing aspirin and an enteric coating layer coated on said core, wherein the difference in the average diameters of said first and second particles is 100 μm to 800 μm; a method for preparing same; and a method of validating the quality of same. The pharmaceutical composite formulation according to the present invention can improve the stability of an active ingredient and prevent adverse impacts between the active ingredients to thereby enable an accurate quality validation of the pharmaceutical composite formulation.

### FIELD OF THE INVENTION

The present invention relates to a pharmaceutical composite formulation comprising an HMG-CoA reductase inhibitor and aspirin for preventing or treating cardiovascular diseases, which has an improved stability and allows an accurate quality validation.

### BACKGROUND OF THE INVENTION

Hyperlipidemia stands for a condition that the levels of lipids such as cholesterols, triglycerides, and others in the plasma are abnormally elevated. Hyperlipidemia, particularly hypercholesterolemia, induces arterial thrombosis, resulting in arteriosclerosis in which an artery wall thickens as a result of accumulation of lipids. Arteriosclerosis is clinically important since it can lead to cardiovascular diseases such as ischemic heart disease, angina pectoris, and myocardial infarction. Arteriosclerosis can be prevented by way of treatment of hypercholesterolemia since the latter is closely highly associated with former.

Hyperlipidemia or an elevated level of lipids in the plasma is related with an increase in the incidence frequency of cardiovascular diseases and arteriosclerosis. More specific types of hyperlipidemia may include hypercholesterolemia, familial dysbetalipoproteinemia, diabetic dyslipidemia, dyslipidemia linked to nephropathy, familial combined hyperlipidemia, and others. Hypercholesterolemia results in elevated levels of low density lipoprotein (LDL)-cholesterol and total cholesterol in the plasma. LDL transports cholesterol in the blood. In addition, familial dysbetalipoproteinemia, also known as type III hyperlipidemia, is characterized by the accumulation of beta VLDL (very low density lipoprotein) in the plasma. Further, this symptom is involved in the replacement of a normal apolipoprotein E3 with an abnormal isoform, apolipoprotein E2. Diabetic dyslipidemia causes a multiple of lipoprotein disorders including overproduction of VLDL-cholesterol, abnormal lipolysis of VLDL triglycerides, decreased activity of LDL-cholesterol receptor, frequently occurring type III hyperlipidemia, and others. Dyslipidemia linked to nephropathy is not readily treated, and its examples that frequently occur may include hypercholesterolemia and hypertriglyceridemia. Familial combined hyperlipidemia is classified into multiple phenotypes of hyperlipidemia, i.e., type IIa, IIb, IV, V or hyperapobetalipoproteinemia.

For decades, HMG-CoA reductase inhibitors have been used to treat hyperlipidemia. These compounds have been known to lower the levels of total cholesterol and LDL-cholesterol in a human body and to elevate the levels of HDL-cholesterol for some individuals. The conversion of HMG-CoA to mevalonate takes place in an early and rate-determining step in the biosynthesis of cholesterol. The inhibition of HMG-CoA reductase, which prevents the production of mevalonate, is correlated with the impact of an HMG-CoA reductase inhibitor on the reduction in total cholesterols and on LDL-cholesterols (see Grundi S. M., N. Engl. J. Med., 319(1): 24-32, 25-26, 31(1988)). Examples of HMG-CoA reductase inhibitors include mevastatin (U.S. Pat. No. 3,983,140), lovastatin (also called mevinolin; U.S. Pat. No. 4,231,938), pravastatin (U.S. Pat. Nos. 4,346,227 and 4,410,629), pravastatin lactone (U.S. Pat. No. 4,448,979), velostatin and simvastatin (also called synvinolin; U.S. Pat. Nos. 4,448,784 and 4,450,171), rivastatin, fluvastatin, atorvastatin, and cerivastatin.

Another mechanism leading to arteriosclerosis is the formation of thrombus. Thrombus is formed by an interaction of platelets and plasma coagulation factors in an injured vessel, which also induces arteriosclerosis. Aspirin is used as an antipyretic to reduce fever, as an analgesic to relieve minor aches and pains, and as an agent to prevent arterial thrombosis. Aspirin (also known as acetylsalicylic acid) irreversibly acetylates cyclooxygenase of platelet, thereby inhibiting the production of thromboxane A2 (TXA2), i.e., a derivative of platelet aggragation, which prevents platelet aggregation in the blood.

Therefore, an HMG-CoA reductase inhibitor and aspirin in combination may be useful for treating various cardiovascular diseases such as hypercholesterolemia and arterial thrombosis. HMG-CoA reductase inhibitors exhibit a poor bioavailability and are absorbed in the gastrointestinal tract. Thus, it would be beneficial for HMG-CoA reductase inhibitors to be rapidly released in the gastrointestinal tract. Meanwhile, aspirin may produce adverse side effects, e.g., gastric ulcer or gastric bleeding when it is released within the gastrointestinal tract, and may interact adversely with HMG-CoA reductase inhibitors when both are released at the same time within the gastrointestinal tract.

According to the U.S. Food and Drug Administration (FDA) Summary Basis of Approval (SBA) for Warner-Lambert's Lipitor™, atorvastatin is present in multiple amorphous and crystalline forms. Originally, atorvastatin was synthesized in an amorphous form, but it has been reported that amorphous atorvastatin is hygroscopic and unstable when exposed to oxygen. On the other hand, a crystalline form of atorvastatin developed later shows an improved in vivo absorption rate (i.e., an approximate 50% increase in Cmax). Nevertheless, it is highly susceptible to heat, moisture, a low pH environment, and light, which requires attention in selecting excipients or additives in its product development.

The stability of an HMG-CoA reductase inhibitor including atorvastatin may be reduced in a low pH environment (i.e., an acidic condition), while aspirin is stable in a low pH condition but unstable in a basic condition. Therefore, a composite formulation comprising both of said drugs may involve a reduced stability caused by the interaction between them.

To solve the problem, the present inventors developed a composite formulation comprising an HMG-CoA reductase inhibitor containing a basic additive, and aspirin coated with an enteric coating layer. However, in a quality validation of the composite formulation, it was observed that aspirin (or aspirin derived salicylic acid) affected the degradation of the HMG-CoA reductase inhibitor in an acidic condition, which prohibited an accurate quality validation of the composite formulation.

Hence, the present inventors have endeavored to develop a composite formulation comprising an HMG-CoA reductase inhibitor and aspirin, and have found that an accurate quality validation of the composite formulation can be assured when said drugs have different particle sizes.

### SUMMARY OF THE INVENTION

Accordingly, it is an object of the present invention to provide a pharmaceutical composite formulation comprising an HMG-CoA reductase inhibitor and aspirin for preventing or treating cardiovascular diseases, which has an improved stability and allows an accurate quality validation.

It is another object of the present invention to provide a method for preparing the pharmaceutical composite formulation.

It is a further object of the present invention to provide an improved method for validating the quality of the pharmaceutical composite formulation.

In accordance with one aspect of the present invention, there is provided a pharmaceutical formulation for preventing or treating cardiovascular diseases, which comprises: (1) a first particle comprising an HMG-CoA reductase inhibitor and a basic additive; and (2) a second particle comprising a core containing aspirin and an enteric coating layer coated on said core, wherein the difference in the average diameters of said first and second particles is 100 μm to 800 μm.

In accordance with another aspect of the present invention, there is provided a method for preparing the pharmaceutical formulation, which comprises the steps of:

(i) preparing a first particle comprising an HMG-CoA reductase inhibitor and a basic additive;

(ii) preparing a second particle comprising a core containing aspirin and an enteric coating layer coated on said core, wherein the difference in the average diameters of said first and second particles is 100 μm to 800 μm; and

(iii) filling a capsule with the first particle and second particles prepared in steps (i) and (ii).

In accordance with a further aspect of the present invention, there is provided a method for validating the quality of the pharmaceutical formulation, which comprises the steps of:

(i) separating a first particle and a second particle from the pharmaceutical formulation; and

(ii) analyzing an impurity of an HMG-CoA reductase inhibitor or aspirin in said first and second particles.

### BRIEF DESCRIPTION OF THE DRAWINGS

The above and other objects and features of the present invention will become apparent from the following description of the invention, when taken in conjunction with the accompanying drawings, in which:

FIG. 1 is a schematic drawing showing the processes of preparing and validating the quality of the pharmaceutical composite formulation according to one embodiment of the present invention; and

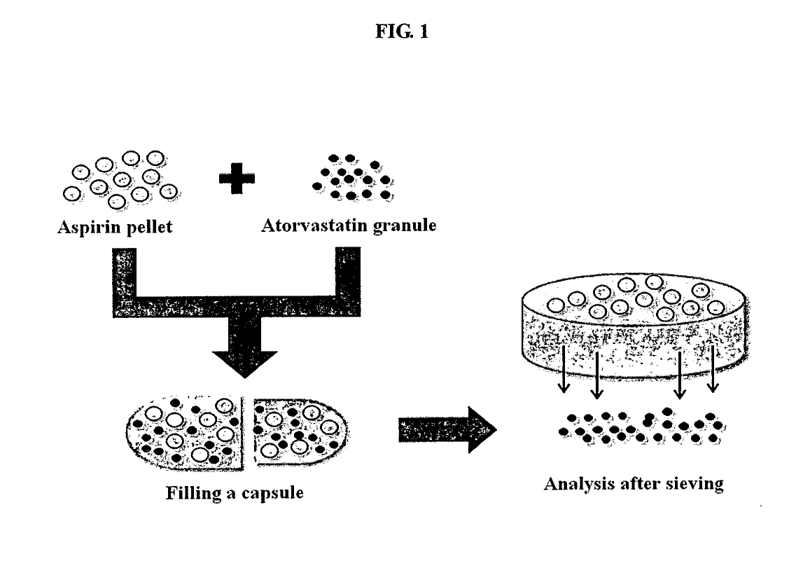

FIG. 2 is a result showing the amounts of atorvastatin lactone (%) in the accelerated conditions after seiving the pharmaceutical composite formulations through various sizes of sieves.

### DETAILED DESCRIPTION OF THE INVENTION

The present invention provides a pharmaceutical composite formulation for preventing or treating cardiovascular diseases, which comprises: (1) a first particle comprising an HMG-CoA reductase inhibitor and a basic additive; and (2) a second particle comprising a core containing aspirin and an enteric coating layer coated on said core, wherein the difference in the average diameters of said first and second particles is 100 μm to 800 μm. Hereinafter, the components contained in the pharmaceutical composite formulation of the present invention are described in detail.

(i) First Particle

The first particle of the pharmaceutical composite formulation according to the present invention contains an HMG-CoA reductase inhibitor as an active ingredient, along with a basic additive.

The HMG-CoA reductase inhibitor is a drug capable of preventing or treating hyperlipidemia and arteriosclerosis by reducing the level of lipoproteins or lipids in the blood. Particular examples thereof may include, but are not limited to, rosuvastatin, lovastatin, atorvastatin, pravastatin, fluvastatin, pitavastatin, simvastatin, rivastatin, cerivastatin, velostatin, mevastatin, and a pharmaceutically acceptable salt, a precursor or a mixture thereof, preferably atorvastatin or a pharmaceutically acceptable salt thereof, more preferably atorvastatin calcium.

The HMG-CoA reductase inhibitors may be used in an amount of about 5 to 35% by weight, preferably about 5 to 25% by weight, based on the total weight of the first particle. Also, the HMG-CoA reductase inhibitor may be used in an amount of 0.5 mg to 100 mg, preferably 5 mg to 80 mg, per a unit dosage form.

The basic additive may be used in the present invention for enhancing the stability of the HMG-CoA reductase inhibitors which are unstable in a low pH condition. Examples thereof may include, but are not limited to, basic minerals such as NaHCO₃, CaCO₃, MgCO₃, KH₂PO₄, K₂HPO₃, tribasic calcium phosphate and others, meglumine, arginine, glysine, aluminium magnesium silicate, aluminium magnesium metasilicate and the like. Preferred is NaHCO₃, CaCO₃, MgCO₃ or a mixture thereof. The basic additives may be used in an amount of 1.5 to 10 parts by weight, based on 1 part by weight of the HMG-CoA reductase inhibitor in the first particle, but its amount is not limited to this range. When the amount of the basic additives is at least 20% by weight, based on the total weight of the first particle, the formation of atorvastatic lactone impurities may be prevented. Thus, the basic additives may be used in an amount of 10 to 70% by weight, preferably 20 to 50% by weight, based on the total weight of the first particle.

In addition, the first particle may further comprise pharmaceutically acceptable water-soluble diluents and optionally other excipients or adjuvants, which may include, but are not limited to, disintegrants, binders, lubricants, coating agents, fillers, rheology modifiers, crystallization retarders, solubilizers, pH modifiers, surfactants, emulsifiers, or a mixture thereof.

Examples of the water-soluble diluents may include glucose, sucrose, lactose, sorbitol, mannitol, dulcitol, ribitol, xylitol, and a mixture thereof, but are not limited thereto. The water-soluble diluents may be used in an amount of 5 to 80% by weight, preferably 5 to 60% by weight, based on the total weight of the first particle.

The disintegrants as the excipients or adjuvants may appropriately be selected from such conventionally available disintegrants as hydroxypropylcellulose, crospovidone, sodium starch glycolate, croscarmellose sodium, and others. The disintegrants may be used in an amount of 5 to 30% by weight, preferably 5 to 20% by weight, based on the total weight of the first particle. The binders as the excipients or adjuvants may include povidone, copovidone, cellulose, and others. The binders may be used in an amount of 0.5 to 5% by weight, preferably 1 to 3% by weight, based on the total weight of the first particle. The lubricants as the excipients or adjuvants may appropriately be selected from such conventionally available lubricants as magnesium stearate, sodium stearyl fumarate, talc, glyceryl fatty acid ester, glycerol dibehenate, and others. The lubricants may be used in an amount of 0.4 to 2% by weight, based on the total weight of the first particle.

In the present invention, the first particle is formulated into powders, granules, pellets, or mini tablets, preferably granules.

(ii) Second Particle

The second particle of the present invention comprises a core containing aspirin and an enteric coating layer coated on the core.

It has been reported that aspirin effectively inhibits platelet aggregation and has a low toxicity, which can be used for treating cardiovascular and cerebrovascular diseases, such as angina pectoris and thromboembolism. Aspirin irreversibly acetylates cyclooxygenase to thereby deactivate it. Cyclooxygenase is essential to the production of thromboxane A2 (TXA2) inducing thrombosis and prostacylclin having an anti-platelet aggregating ability. Aspirin in a low amount plays a role in keeping the synthesis of cyclooxygenase and prostacylclin in endotheliocytes, while selectively inhibiting cyclooxygenase in platelet, thereby reducing inflammation, platelet aggregation and thrombosis in the blood.

Aspirin may be used in an amount of 10 mg to 2 g per the formulation, which is effective in treating the inhibition of platelet aggregation. It may be employed in an amount of about 1 to 80% by weight, preferably about 5 to 60% by weight, based on the total weight of the second particle, but its amount is not limited to these ranges.

The core containing aspirin may further comprise anti-platelet aggregating agents including salicylates such as salicylate magnesium, anagrelide, dipyridamole, clopidogrel, and ticlopidin (see U.S. Pat. No. 7,002,962), and acidifying materials for stabilizing aspirin including citric acid, alginic acid, glutamic acid and the like (see U.S. Pat. No. 4,716,042), but being not limited thereto.

The enteric coating layer of the present invention may be formed on the surface of the core containing aspirin to protect aspirin from gastric fluid and to release aspirin in the intestine. Also, the enteric coating layer may prevent the HMG-CoA reductase inhibitor contained in the first particle from interacting with aspirin, thereby improving the stability of the HMG-CoA reductase inhibitor. It also may prevent the basic additives contained in the first particle from interacting with aspirin, thereby improving the stability of aspirin. The enteric coating layer may comprise conventional enteric coating layer-forming materials or coating layer-forming materials for delayed release. Examples of the enteric coating layer-forming materials may include hydroxypropyl methylcellulose phthalate (HPMCP), hydroxypropyl methylcellulose, methacrylic acid, cellulose acetate phthalate (CAP) and the like. Examples of the coating layer-forming materials for delayed release may include ethyl cellulose, cellulose acetate, polyvinyl acetate and the like. The enteric coating layer-forming materials or the coating layer-forming materials for delayed release of the present invention may be used in an amount of 0.05 to 0.6 parts by weight, preferably 0.1 to 0.5 parts by weight, based on 1 part by weight of aspirin, and may be used in an amount of about 10 to 50% by weight, preferably 10 to 30% by weight, based on the total weight of the second particle, but their amounts are not limited to these ranges.

In addition, the enteric coating layer may further comprise conventional pharmaceutically acceptable excipients, such as plasticizers and lubricants, for adhering the enteric coating layer-forming materials to aspirin. Examples of the pharmaceutically acceptable plasticizers may include glycerin, propylene glycol, sorbitol, maltitol, mannitol, a mixture of hydrated starch lydrolysates and the like, and may appropriately be selected from conventionally available plasticizers. The plasticizers may be used in an amount of about 0.5 to 5% by weight, preferably about 1 to 3% by weight, based on the total weight of the second particle. Examples of the pharmaceutically acceptable lubricants may include calcium stearate, glyceryl monostearate, glyceryl palmitostearate, magnesium stearate, sodium lauryl sulfate, sodium stearyl fumarate, zinc stearate, stearate, hydrogenated vegetable oil, polyethylene glycol, sodium benzoate, talc and the like, and may appropriately be selected from conventionally available lubricants. The lubricants may be used in an amount of about 0.4 to 10% by weight, preferably 0.4 to 5% by weight, based on the total weight of the pharmaceutical composite formulation.

(iii) Hydrophobic Coating Layer

The second particle of the present invention may further comprise a hydrophobic coating layer in addition to the enteric coating layer formed on the surface of the core containing aspirin. The hydrophobic coating layer of the present invention is used for the purpose of preventing salicylic acid released from aspirin from moving toward the first particle containing the HMG-CoA reductase inhibitors, which is different from the function of the enteric coating layer for releasing aspirin in a pH dependent manner.

Examples of the hydrophobic coating layer-forming materials may include waxes such as carnauba wax, glyceryl monostearate, glyceryl monooleate and beeswax; and synthetic or semi-synthetic hydrophobic polymers such as ethyl cellulose, aminoalkyl methacrylate copolymer RS, ethyl acrylate-methyl methacrylate copolymer, polyvinyl chloride, polyvinyl acetate and cellulose acetate.

The hydrophobic coating layer may further comprise plasticizers such as triethyl citrate, polyethylene glycol, propylene glycol, acetylated monoglyceride, diethyl phthalate, dibutyl sebacate and the like, and may also comprise additional coating bases commonly used in pharmaceutical industry such as HPMC, HPC, polyvinyl alcohol, and the like. In addition, talc, titanium dioxide, and the like may be used to prevent adhesion of pellets during the coating procedure.

(iv) Difference in Average Diameter of Particles

In the pharmaceutical composite formulation according to the present invention, the first and second particles may be separated from each other owing to the different average diameters of the particles.

A conventional composite formulation generally has difference ranging from 5 μm to 75 μm in the particle diameters of the active ingredients. If the active ingredients contained in the composite formulation do not interact with each other or such interaction is trivial, there would be no problem. If the active ingredients contained in the composite formulation are highly interactive with each other, however, it would produce an inaccurate result in the quality validation of the composite formulation. For example, aspirin or salicylic acid derived therefrom as one active ingredient of the pharmaceutical composite formulation according to the present invention adversely affects the HMG-CoA reductase inhibitor as another active ingredient, thereby increasing the amount of impurities of the HMG-CoA reductase inhibitor generated during the quality validation, which prohibits an accurate quality validation of the pharmaceutical composite formulation.

The pharmaceutical composite formulation of the present invention is designed such that the first particle containing an HMG-CoA reductase inhibitor and the second particle containing aspirin have different average particle diameters, wherein the difference is in the range of 100 μm to 800 μm, preferably 200 μm to 800 μm. Due to such difference in their average particle diameters, the first and second particles can physically be separated. If the difference in the average particle diameters is less than 100 μm, the first and second particles may not be physically separated. If it exceeds 800 μm, the first and second particles may not be filled in a capsule.

The difference in the particle diameters as stated above, enables each active ingredient to be easily separated in the quality validation process necessary for the preparation of medicines. In particular, in an analysis of the stability of medicines, physical separation of the first and second particles can minimize the generation of impurities, which assures an accurate quality validation. For an accurate quality validation, the first and second particles may be physically separated in an amount of at least 90%, preferably at least 95%, for example using a sieve.

(v) Filling a Capsule

In one preferred embodiment of the present invention, the pharmaceutical composite formulation of the present invention, which comprises a first particle containing an HMG-CoA reductase inhibitor and the second particle containing aspirin may be filled to a capsule.

The present invention also provides a method for preparing the pharmaceutical composite formulation, which comprises the steps of: (i) preparing a first particle comprising an HMG-CoA reductase inhibitor and a basic additive; (ii) preparing a second particle comprising a core containing aspirin and an enteric coating layer coated on said core, wherein the difference in the average diameters of said first and second particles is 100 μm to 800 μm; and (iii) filling a capsule with the first particle and second particles prepared in steps (i) and (ii).

Each step in the preparation of the pharmaceutical composite formulation of the present invention may be carried out in accordance with conventional techniques known in the pharmaceutical industry.

In one embodiment of the present invention, step (i) may be performed by dissolving an HMG-CoA reductase inhibitor, a basic additive and pharmaceutically acceptable excipients in water, drying and granulating the mixture to obtain a granule of the HMG-CoA reductase inhibitor.

In one preferred embodiment of the present invention, step (i) may comprise the following steps:

(a) blending atorvastatin with a preferred disintegrant and optionally some or all of excipients necessary for a final composition. The excipient may include diluents, binders, and other substances necessary for improving the fluidity and stability or processing and formation of unit dosage forms;

(b) adding a granulization solvent to the mixture obtained in step (a) under shear conditions. Preferably, the granulization solvent may include water, ethanol, isopropanol, and a mixture thereof. Other additives (e.g., binders, wetting agents, buffers, etc.) known in the art may be added to the granulization solvent. Various methods known in the art, based on high shear granulation, low shear granulation, fluidized bed granulation, extrusion granulation, and others, may be used in step (b). Also, the granule that has passed through an extruder may further be formed in a spherical shape by a spheronization process for improving the fluidity of the granule;

(c) optionally, pulverizing, milling, or sieving the resultant obtained in step (b), followed by drying the wet material through air drying, fluidized bed drying, oven drying or microwave drying; and

(d) blending the composition thus obtained in step (c) with one or more disintegrants, and optionally additional excipients preferably including lubricants.

In one embodiment of the present invention, step (ii) may be performed by coating the surface of the core containing aspirin with a coating solution comprising enteric coating layer-forming materials, and drying it to obtain an aspirin pellet having an enteric coating layer on its surface.

Also, in one preferred embodiment of the present invention, step (ii) may comprise the following steps:

(a) preparing a spherical aspirin particle by an extrusion spheronization granulation process or a bead coating process. Diluents, binders, and other substances necessary for improving the fluidity and stability or processing and formation of unit dosage forms may be employed;

(b) adding an enteric coating solvent to the particle obtained in step (a) under shear conditions. Preferably, the enteric coating solvent may include water, ethanol, acetone, isopropanol, and a mixture thereof. Other additives (e.g., enteric coating layer-forming materials, binders, wetting agents, buffers, etc.) known in the art may be added thereto. Various methods known in the art, based on high shear granulation, low shear granulation, fluidized bed granulation, extrusion granulation, and others, may be used in step (b); and

(c) drying the wet material obtained in step (b) through air drying, fluid bed drying, oven drying or microwave drying.

Step (ii) involves controlling the difference in the average particle diameters of the first and second particles to produce the effects disclosed in the specification. In one embodiment of the present invention, when the first and second particles are in the form of pellet, the final pellet size may be controlled by adjusting the size of microcrystalline spherical beads, which are commercially available or can be prepared, to a desired size. In another embodiment of the present invention, when the first and second particles are in the form of granule, the grain size may be controlled by employing mills or sieves. In a further embodiment of the present invention, when the first and second particles are in the form of tablet, the tablet size may be controlled by adjusting the size of a punch to be employed. The grain size may be controlled such that an average diameter of the first particle is larger than that of the second particle by 100 μm to 800 μm, or an average diameter of the second particle is larger than that of the first particle by 100 μm to 800 μm.

The present invention also provides a method for validating the quality of the pharmaceutical composite formulation, which comprises the steps of: (i) separating a first particle and a second particle from the pharmaceutical composite formulation; and (ii) analyzing an impurity of an HMG-CoA reductase inhibitor or aspirin in said first and second particles.

The method for validating the quality of the pharmaceutical composite formulation according to the present invention may preferably be performed by physically separating the first particle containing an HMG-CoA reductase inhibitor and the second particle containing aspirin, followed by analyzing the impurities. In one embodiment of the present invention, in case of analyzing the impurities of the first particle containing an HMG-CoA reductase inhibitor in step (ii), it is preferred that the second particle containing aspirin is present in an amount of at most 10% by weight. Preferably, such physical separation may be carried out by using, e.g., sieves. The sieves having a mesh of 40 to 60 are preferred. In case a sieve having a mesh of less than 40 is used, both of the first and second particles are all sieved, prohibiting the separation of the particles, In case a sieve having a mesh of more than 60 is used, both of the first and second particles are not sieved, prohibiting the separation of the particles as well. The method for validating the quality according to the present invention may reduce the adverse impacts of aspirin on the HMG-CoA reductase inhibitor during the quality validation process, which provides a more accurate result on the stability of the medicine to be tested.

The following Examples are provided to illustrate preferred embodiments of the invention, and are not intended to limit the scope of the present invention.

### PREPARATION EXAMPLE 1

### Preparation of Atorvastatin Granules

In accordance with the ingredients described in Table 1, atorvastatin calcium (Dr. Reddy's Laboratories Ltd., India), croscarmellose sodium (DMV International) and magnesium carbonate (Tomita, Japan) were mixed. The mixture was then kneaded with a binding solution of HPC and polysorbate 80 (Croda, USA) dissolved in water, dried, and subsequently sieved through a sieve having a mesh of 40 (passable size of particle, screen size 425 μm) to obtain wet granules, followed by admixing the resultant with magnesium stearate to prepare atorvastatin granules. The average particle size of the granules thus obtained was determined by particle size image analyzer (Qicpic, Sympatec, X50), which was 400 μm.

TABLE 1
Ingredients of atorvastatin granules (unit: mg)

| Ingredients | Prep. Ex. 1 |
|---|---|
| Atorvastatin calcium | 21.69 |
| Magnesium carbonate | 40 |
| Croscarmellose sodium | 18 |
| HPC | 3 |
| Polysorbate 80 | 1.2 |
| <Water> | <80> |
| Magnesium stearate | 0.5 |
| Total | 86.19 |
| Average particle diameter | 400 μm |

### PREPARATION EXAMPLE 2

### Preparation of 1^{st} Aspirin Pellets

In accordance with the ingredients described in Table 2, aspirin (Spectrum Chemical, USA), hydroxypropylmethyl cellulose (HPMC)(Shinetsu, Japan), citric acid, polyethylene glycol and talc were dissolved in a mixture of water and acetone to obtain a coating solution containing aspirin. The coating solution thus obtained was sprayed to microcrystalline spherical beads (Pharmatrans Sanaq, Switherland) in a fluidized bed coating machine (NQ-125, Fujipaudal, Japan) to obtain pellets containing aspirin. In Preparation Examples 2-1 to 2-3, pellets having various particle diameters were prepared by employing MCC101 (bead diameter 50 μm), MCC102 (bead diameter 100 μm) and MCC200 (bead diameter 180 μm) as the microcrystalline spherical beads, respectively.

TABLE 2
Ingredients of 1^{st} aspirin pellets (unit: mg)

| Ingredients | Prep. Ex. 2-1 | Prep. Ex. 2-2 | Prep. Ex. 2-3 |
|---|---|---|---|
| Microcrystalline | 37.5 | 37.5 | 37.5 |
| spherical bead | (MCC101: | (MCC102: | (MCC200: |
| (type: bead diameter) | 50 μm) | 100 μm) | 180 μm) |
| Aspirin | 100 | 100 | 100 |
| Citric acid | 10 | 10 | 10 |
| HPMC | 10 | 10 | 10 |
| Polyethylene glycol | 1 | 1 | 1 |
| Talc | 1 | 1 | 1 |
| <Water> | <100> | <100> | <100> |
| <acetone> | <500> | <500> | <500> |
| Total | 159.5 | 159.5 | 159.5 |

### PREPARATION EXAMPLE 3

### Preparation of 2^{nd} Aspirin Pellets having an Enteric Coating Layer

In accordance with the ingredients described in Table 3, the 1^{st} aspirin pellets prepared in Preparation Example 2 were coated with an enteric coating layer. Hydroxypropylmethyl cellulose phthalate (HPMCP) (Shinetsu, Japan), Myvacet, talc and TiO₂ were dissolved and dispersed in a mixture of ethanol and acetone, to prepare an enteric coating solution. The enteric coating solution was sprayed to the 1^{st} aspirin pellets prepared in Preparation Example 2 in a fluidized bed coating machine (NQ-125, Fujipaudal, Japan). The resultant was then dried to obtain pellets having the enteric coating layer (Preparation Examples 3-1 to 3-3).

TABLE 3
Ingredients of 2^{nd} aspirin pellets having an enteric coating layer (unit: mg)

| Ingredients | Prep. Ex. 3-1 | Prep. Ex. 3-2 | Prep. Ex. 3-3 |
|---|---|---|---|
| 1^{st} aspirin pellets | Prep. Ex. 2-1 | Prep. Ex. 2-2 | Prep. Ex. 2-3 |
| | 159.5 | 159.5 | 159.5 |
| HPMCP | 39.5 | 39.5 | 39.5 |
| Myvacet | 1.9 | 1.9 | 1.9 |
| Talc | 5.7 | 5.7 | 5.7 |
| TiO₂ | 0.9 | 0.9 | 0.9 |
| <Ethanol> | <114.5> | <114.5> | <114.5> |
| <Acetone> | <335> | <335> | <335> |
| Total | 207.5 | 207.5 | 207.5 |

### PREPARATION EXAMPLE 4

### Preparation of Final Aspirin Pellets having a Hydrophobic Film

In accordance with the ingredients described in Table 4, the pellets having the enteric coating layer prepared in Preparation Example 3 were coated with a hydrophobic film. Hydroxypropylmethyl cellulose (HPMC)(Shinetsu, Japan), ethylcellulose (Dow chemical, USA), polyethylene glycol, talc, TiO₂ and iron oxide yellow were dissolved and dispersed in a mixture of water and ethanol to obtain a hydrophobic film coating solution. The hydrophobic film coating solution thus obtained was sprayed to the pellets having the enteric coating layer prepared in Preparation Example 3 in a fluidized bed coating machine (NQ-125, Fujipaudal, Japan). The resultant was then dried to obtain final aspirin pellets having the hydrophobic film. The average particle diameter of the pellets thus obtained was determined by a particle size image analyzer (Qicpic, Sympatec, X50). The results are shown in Table 4.

TABLE 4
Ingredients of aspirin pellets having hydrophobic film (unit: mg)

| Ingredients | Prep. Ex. 4-1 | Prep. Ex. 4-2 | Prep. Ex. 4-3 |
|---|---|---|---|
| 2^{nd} enteric aspirin pellets | Prep. Ex. 3-1 | Prep. Ex. 3-2 | Prep. Ex. 3-3 |
| | 207.5 | 207.5 | 207.5 |
| HPMC | 11.0 | 11.0 | 11.0 |
| Ethyl cellulose | 4.8 | 4.8 | 4.8 |
| Polyethylene glycol | 1.7 | 1.7 | 1.7 |
| Talc | 4.5 | 4.5 | 4.5 |
| TiO₂ | 0.6 | 0.6 | 0.6 |
| Iron oxide yellow | 0.6 | 0.6 | 0.6 |
| <Water> | <78.3> | <78.3> | <78.3> |
| <Acetone> | <156.7> | <156.7> | <156.7> |
| Total | 230.7 | 230.7 | 230.7 |
| Average particle diameter | 405 μm | 475 μm | 590 μm |

### EXAMPLE 1

### Preparation of a Composite Formulation Comprising Atorvastatin Granules and Aspirin Pellets

20 mg of the atorvastatin granule prepared in Preparation Example 1 and 100 mg of the aspirin pellet prepared in Preparation Example 4-3 were mixed. The mixture was filled to a capsule to obtain a composite formulation of Example 1 as set forth in Table 5. The difference in the average particle diameters of the atorvastatin granule and the aspirin pellet of the composite formulation of Example 1 was 190 μm.

### COMPARATIVE EXAMPLES 1 TO 3

### Preparation of a Single or Composite Formulation Comprising Atorvastatin Granules and/or Aspirin Pellets

A single formulation of Comparative Example 1 was prepared from the atorvastatin granule prepared in Preparation Example 1. Composite formulations of Comparative Examples 2 and 3 were prepared from the aspirin pellet of Preparation Examples 4-1 and 4-2, respectively, instead of the aspirin pellet Preparation Example 4-3. The differences in the average particle diameters of the atorvastatin granule and the aspirin pellet of these composite formulations of Comparative Examples 2 and 3 were 5 μm and 75 μm, respectively.

TABLE 5
Comparison between the formulations of
Comparative Examples and Example

| | Com. Ex. 1 | Com. Ex. 2 | Com. Ex. 3 | Ex. 1 |
|---|---|---|---|---|
| Atorvastatin | Prep. Ex. 1 | Prep. Ex. 1 | Prep. Ex. 1 | Prep. Ex. 1 |
| granule | | | | |
| Aspirin pellet | — | Pre. Ex. 4-1 | Pre. Ex. 4-2 | Pre. Ex. 4-3 |
| Atorvastatin | 400 μm | 400 μm | 400 μm | 400 μm |
| granule | | | | |
| Aspirin pellet | — | 405 μm | 475 μm | 590 μm |
| Difference in | — | 5 μm | 75 μm | 190 μm |
| particle diameter | | | | |

### EXPERIMENTAL EXAMPLE 1

### Stability Evaluation of the Composite Formulations

### <1-1> Physical Separation of Composite Formulations

The particles of the composite formulations prepared in Example 1, and Comparative Examples 2 and 3 were sieved through sieves having meshes of 25, 30, 35, 40, 45 and 60. The amount of the atorvastatin and aspirin particles that passed through the sieves was measured. The results are shown in Table 6.

TABLE 6
Amount of atorvastatin and aspirin that passed through the sieves

| Sieve | 25 mesh | | | 30 mesh | | | 35 mesh | | | 40 mesh | | | 45 mesh | | | 60 mesh | | |
|---|---|---|---|---|---|---|---|---|---|---|---|---|---|---|---|---|---|---|
| | Com. | Com. | | Com. | Com. | | Com. | Com. | | Com. | Com. | | Com. | Com. | | Com. | Com. | |
| Sample | Ex. 2 | Ex. 3 | Ex. 1 | Ex. 2 | Ex. 3 | Ex. 1 | Ex. 2 | Ex. 3 | Ex. 1 | Ex. 2 | Ex. 3 | Ex. 1 | Ex. 2 | Ex. 3 | Ex. 1 | Ex. 2 | Ex. 3 | Ex. 1 |
| Atorvastatin | 100% | 100% | 100% | 100% | 100% | 100% | 95% | 95% | 95% | 90% | 90% | 90% | 70% | 70% | 70% | 15% | 15% | 15% |
| Aspirin | 100% | 100% | 100% | 100% | 95% | 90% | 95% | 90% | 45% | 90% | 35% | 10% | 70% | 5% | 0% | 20% | 0% | 0% |

As shown in Table 6, the composite formulation of Example 1 wherein the difference in the average particle diameters was 190 μm represented that the amounts of atorvastatin and aspirin that passed through the sieve having a mesh of 40 were above 90% by weight and below 10% by weight. Thus, the composite formulation of Example 1 was physically separated.

Meanwhile, the composite formulation of Comparative Example 1 wherein the difference in the average particle diameters was 5 μm was not physically separated even with sieves having meshes of 25 to 60. Also, the composite formulation of Comparative Example 3 wherein the difference in the average particle diameters was 75 μm was not physically separated with a sieve having a mesh of 60. Although the amount of the aspirin pellets was 0% by weight that of the atorvastatin granules was also very low.

These results demonstrate that the difference in the average particle diameters of the atorvastatin and aspirin particles must be at least 100 μm in order for them to be physically separated.

### <1-2> Impurity Analysis of Composite Formulations

In accordance with the method for analyzing atorvastatin impurities described below, test solutions were prepared. The amounts of atorvastatin lactone impurities of the composite formulations sieved in Experimental Example 1-1 and Comparative Example 1 were determined.

[Method for Analyzing Atorvastatin Impurities]

100 mg of atorvastatin was added to a 200 ml flask, 150 ml of a dilutent solution was added thereto, and a dilutent solution was further added to make 200 ml. The mixture was filtered through a 0.45 μm membrane filter, and the filtrate thus obtained served as a test solution. The impurities were measured by comparing the relative retention time (RRT) detected under the following conditions.
Detector: UV-VIS spectro-photometer (254 nm)
Column: Column having a stainless pipe filled with 5 μm C18 (inner diameter of 4 6 mm×length of about 250 mm), or similar one
Dilutent: 60:5:35 (Acetonitrile:tetrahydrofuran:water)
Equation: Impurity (%)=(Peak area of each impurity/sum of peak areas except for solvent peak)×(100/RRF)

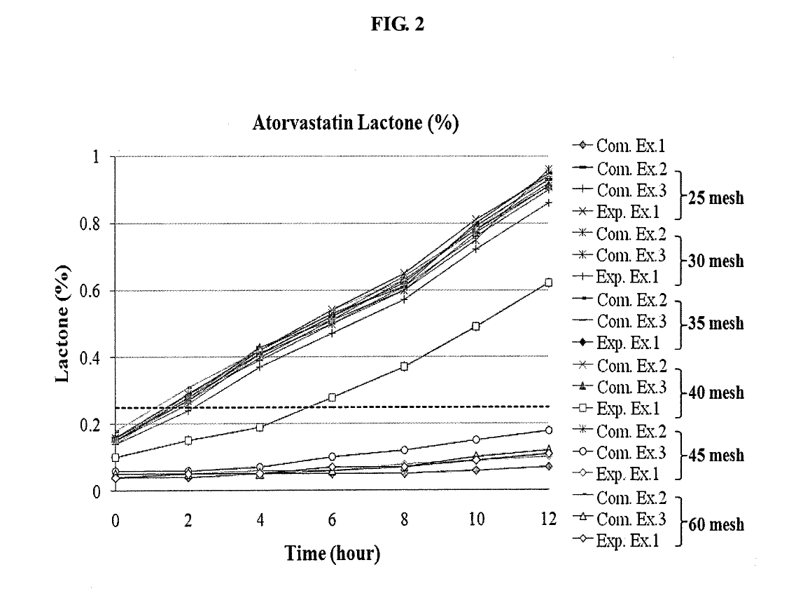

The results are shown in Table 7 and FIG. 2.

TABLE 7
Amounts of atorvastatin lactone impurities in the filtrates with time

| Sieve | | 25 mesh | | | 30 mesh | | | 35 mesh | | | 40 mesh | | | 30 mesh | | | 35 mesh | | |
|---|---|---|---|---|---|---|---|---|---|---|---|---|---|---|---|---|---|---|---|
| | Com. | Com. | Com. | Exp. | Com. | Com. | Exp. | Com. | Com. | Exp. | Com. | Com. | Exp. | Com. | Com. | Exp. | Com. | Com. | Exp. |
| Sample | Ex. 1 | Ex. 2 | Ex. 3 | Ex. 1 | Ex. 2 | Ex. 3 | Ex. 1 | Ex. 2 | Ex.3 | Ex. 1 | Ex. 2 | Ex. 3 | Ex. 1 | Ex. 2 | Ex. 3 | Ex. 1 | Ex. 2 | Ex.3 | Ex. 1 |
| Hour | Lactone | | | | | | | | | | | | | | | | | | |
| 0 | 0.04 | 0.15 | 0.14 | 0.16 | 0.15 | 0.15 | 0.14 | 0.16 | 0.16 | 0.10 | 0.17 | 0.10 | 0.08 | 0.16 | 0.06 | 0.04 | 0.18 | 0.05 | 0.04 |
| 2 | 0.04 | 0.28 | 0.29 | 0.27 | 0.27 | 0.26 | 0.24 | 0.29 | 0.28 | 0.15 | 0.30 | 0.14 | 0.09 | 0.29 | 0.06 | 0.05 | 0.31 | 0.05 | 0.05 |
| 4 | 0.05 | 0.41 | 0.39 | 0.42 | 0.40 | 0.41 | 0.37 | 0.42 | 0.43 | 0.19 | 0.43 | 0.17 | 0.11 | 0.41 | 0.07 | 0.06 | 0.42 | 0.05 | 0.05 |
| 6 | 0.05 | 0.52 | 0.50 | 0.54 | 0.51 | 0.50 | 0.47 | 0.53 | 0.51 | 0.28 | 0.54 | 0.26 | 0.15 | 0.52 | 0.10 | 0.06 | 0.54 | 0.06 | 0.07 |
| 8 | 0.05 | 0.62 | 0.61 | 0.65 | 0.60 | 0.60 | 0.57 | 0.61 | 0.63 | 0.37 | 0.64 | 0.34 | 0.18 | 0.64 | 0.12 | 0.08 | 0.63 | 0.07 | 0.07 |
| 10 | 0.06 | 0.79 | 0.76 | 0.81 | 0.75 | 0.77 | 0.72 | 0.80 | 0.78 | 0.49 | 0.81 | 0.45 | 0.21 | 0.78 | 0.15 | 0.09 | 0.78 | 0.10 | 0.09 |
| 12 | 0.07 | 0.93 | 0.90 | 0.94 | 0.96 | 0.91 | 0.86 | 0.95 | 0.91 | 0.62 | 0.95 | 0.59 | 0.24 | 0.92 | 0.18 | 0.10 | 0.94 | 0.12 | 0.11 |

Whether the impurities are increased in the filtrates or not is an important factor for assuring the accuracy and stability in drug quality validations. The standard amount of the representative impurity, atorvastatin lactone, is less than 0.25% by weight according to the standard concentrate limits described in the ICH (International Conference on Harmonization) guideline.

As shown in Table 7 and FIG. 2, the more aspirin passed through the sieves, the more atorvastatin lactone was detected with time. In contrast, in case substantially no aspirin remained in the composite formulation since it did not pass through the sieves, the amount of atorvastatin lactone did not be increased with time. In particular, the result for the composite formulation of Example 1 sieved with a 40 mesh sieve shows that the amount of atorvastatin lactone met the standard concentrate limit when aspirin was in an amount of less than about 10% by weight. The results suggest that only when atorvastatin contains less than 10% by weight of aspirin, i.e., at least 90% by weight of aspirin is filtered out, an accurate analysis of the atorvastatin impurities can be guaranteed.

In other words, at least 90% by weight of aspirin must be separated from atorvastatin by a physical separation for an accurate analysis of the atorvastatin impurities.

Also, in the pharmaceutical composite formulation comprising atorvastatin and aspirin, the difference in the average particle diameters of the particles containing the two active ingredients must be in the range of at least 100 μm, preferably 200 μm, so that at least 90% by weight of atorvastatin particles can be separated from aspirin particles. In such case, the problem of stability of atorvastatin during its impurity analysis may be solved.

While the invention has been described with respect to the above specific embodiments, it should be recognized that various modifications and changes may be made to the invention by those skilled in the art which also fall within the scope of the invention as defined by the appended claims.

What is claimed is:

1. A pharmaceutical formulation for preventing or treating cardiovascular diseases, which comprises:
(1) a first particle comprising an HMG-CoA reductase inhibitor and a basic additive; and
(2) a second particle comprising a core containing aspirin and an enteric coating layer coated on said core,
wherein the difference in the average diameters of said first and second particles is 100 μm to 800 μm.

2. The pharmaceutical formulation of claim 1, wherein said HMG-CoA reductase inhibitor is selected from the group consisting of rosuvastatin, lovastatin, atorvastatin, pravastatin, fluvastatin, pitavastatin, simvastatin, rivastatin, cerivastatin, velostatin, mevastatin, and a pharmaceutically acceptable salt, a precursor and a mixture thereof.

3. The pharmaceutical formulation of claim 1, wherein said HMG-CoA reductase inhibitor is atorvastatin calcium.

4. The pharmaceutical formulation of claim 1, wherein said basic additive is selected from the group consisting of NaHCO₃, CaCO₃, MgCO₃, KH₂PO₄, K₂HPO₃, tribasic calcium phosphate, meglumine, arginine, glysine, aluminum magnesium silicate, aluminum magnesium metasilicate, and a mixture thereof.

5. The pharmaceutical formulation of claim 1, wherein said first particle is formulated into powder, granule, pellet, or mini tablet.

6. The pharmaceutical formulation of claim 5, wherein said first particle is formulated into granule.

7. The pharmaceutical formulation of claim 1, wherein said second particle is formulated into granule, pellet, or mini tablet.

8. The pharmaceutical formulation of claim 7, wherein said second particle is formulated into pellet.

9. The pharmaceutical formulation of claim 1, wherein said enteric coating layer comprises one selected from the group consisting of hydroxypropyl methylcellulose phthalate (HPMCP), methacrylic acid, cellulose acetate phthalate (CAP), ethylcellulose, cellulose acetate, polyvinyl acetate and a mixture thereof.

10. The pharmaceutical formulation of claim 1, wherein said second particle further comprises a hydrophobic coating layer comprising a hydrophobic additive, wherein said hydrophobic coating layer is formed on the surface of said enteric coating layer.

11. The pharmaceutical formulation of claim 10, wherein said hydrophobic additive is ethyl cellulose (EC).

12. The pharmaceutical formulation of claim 1, wherein the difference in the average diameters of said first and second particles is 200 μm to 800 μm.

13. A method for preparing said pharmaceutical formulation of claim 1, which comprises the steps of:
(i) preparing a first particle comprising an HMG-CoA reductase inhibitor and a basic additive;
(ii) preparing a second particle comprising a core containing aspirin and an enteric coating layer coated on said core, wherein the difference in the average diameters of said first and second particles is 100 μm to 800 μm; and
(iii) filling a capsule with said first and second particles prepared in steps (i) and (ii).

14. A method for validating the quality of said pharmaceutical formulation of claim 1, which comprises the steps of:
(i) separating a first particle and a second particle from said pharmaceutical formulation; and
(ii) analyzing an impurity of an HMG-CoA reductase inhibitor or aspirin in said first and second particles.
